(19) United States
(12) Patent Application Publication
(10) Pub. No.: US 2023/0397611 A1
(43) Pub. Date: Dec. 14, 2023

(54) Strain of the entomopathogenic fungus beauveria bassiana for protecting agricultural crops against crop-destroying insects and ticks

(71) Applicant: Organic park limited liability company, Kazan (RU)

(72) Inventors: Anna Alexandrovna EGORSHINA, Kazan (RU); Mikhail Alexandrovich LUKYANTSEV, Kazan (RU); Darya Yuryevna NAZARENKO, Kazan (RU)

(73) Assignee: Organic park limited liability company, Kazan (RU)

(21) Appl. No.: 18/027,353

(22) PCT Filed: Aug. 16, 2021
(86) PCT No.: PCT/RU21/50263

§ 371 Date: Mar. 20, 2023

(30) Foreign Application Priority Data
Sep. 21, 2020 (RU) 2020131207

(51) Int. Cl. A01N 63/30; C12N 1/14; A01P 7/04; A01P 7/02

(52) U.S. Cl. CPC A01N 63/30; C12N 1/145; C12R 2001/645; A01P 7/02; A01P 7/04

(57) ABSTRACT

The proposed solution relates to agricultural microbiology and biotechnology. A strain of the entomopathogenic fungus Beauveria bassiana 17HA has been deposited with the Russian National Collection of Industrial Microorganisms under number VKPM F-1526. The VKPM F-1526 strain of the entomopathogenic fungus Beauveria bassiana can be used to create a broad-spectrum microbiological insecticide suitable for treating different agricultural crops. The use of the strain makes it possible to increase crops yield.

### CROSS-REFERENCE TO RELATED APPLICATIONS

This application is a U.S. national stage application of an international application PCT/RU2021/050263 filed on 16 Aug. 2021, published as WO/2022/060252, which international application claims priority of a Russian Federation patent application RU2020131207 filed on 21 Sep. 2020.

### FIELD OF THE INVENTION

The invention relates to agricultural microbiology and biotechnology, relates to biological agents for the protection of agricultural plants against insects and ticks—pests and is a strain of the entomopathogenic fungus Beauveria bassiana 17HA, deposited in the National Bioresource Center All-Russian Collection of Industrial Microorganisms NRC “Kurchatov Institute”—GosNIIGenetika (address: 1st Dorozhny proezd, 1, 117545 Moscow) under registration number VKPM F-1526, deposited date: 19 Aug. 2020.

### BACKGROUND OF THE INVENTION

The use of entomopathogenic microorganisms to control insect pests of agricultural plants appears to be a promising alternative to chemical insecticides. The use of entomopathogenic microorganisms to control insect pests of agricultural plants appears to be a promising alternative to chemical insecticides. In this aspect, microscopic fungi, compared with bacteria and viruses, are interesting, mainly for two reasons: 1) the lack of a narrow specialization in relation to target insects in most strains; 2) to start the infectious process, it is enough for the spores of the fungus to get on the outer integument of the insect. At the first stage of the infectious process, the fungal conidia adhere to the insect cuticle through nonspecific hydrophobic and electrostatic interactions. The conidia then germinates, in most cases forming an appressorium that ruptures the cuticle. As soon as the fungus grows to the hemocoel, its cells acquire a yeast-like form (the so-called hyphal bodies), which gradually infect the host tissues, eventually leading to the mummification of the insect. These cells can also produce secondary metabolites with immunosuppressive activity that enhance the development of fungal infection. A mummified insect is a source of infection for other insects, which in some cases can lead to epizootics.

Among entomopathogenic fungi, representatives of the genera Beauveria and Metarhizium have found the most widespread use as the basis of microbiological insecticides.

There is known strain Beauveria bassiana CL 67-13n (a.c. USSR 572163), obtained as the result of directed changes in the virulence of a natural isolate, exhibiting high virulence against forestry pests.

There is known strains of Beauveria bassiana 113H-278 (a.c. USSR 507645), obtained by selection based on natural monospore sieving after passage through the Colorado potato beetle, and Beauveria bassiana VILL 476-4C (a.c. USSR 982630), obtained by step selection under the action of nitromethylbiuret from strain 113H-278, which has a higher productivity and virulence, intended for use as a producer of the preparation of boverin for the control of the Colorado potato beetle.

There is known strain Beauveria bassiana VKPM G-399 (a.c. USSR 1688820) for the production of insecticidal preparation of boverin against pests of agriculture and forestry. The activity of the strain was detected against the Colorado potato beetle (larvae and adults), grain grinder, barn weevil, rice weevil, red flour beetle.

There is known strain Beauveria bassiana 701 (USSR patent 1795980) to obtain entomopathogenic drug. Activity has been described against greenhouse whitefly on cucumber and larvae of larch fly in the forest floor under the crown of larch.

There is known strain of Beauveria bassiana CK MK “B” N CKM F-54C (patent of the Russian Federation 2034469) to obtain an entomopathogenic drug with increased productivity and thermal resistance. Among insects sensitive to it are thrips, greenhouse whitefly and spider mite.

There is known strain of Beauveria bassiana ATCC N 74040 (patent of the Russian Federation 2103873) for obtaining an entomopathogenic drug against cotton weevil and cotton gadfly.

There is known strain Beauveria bassiana VKM F-3732 D (patent of the Russian Federation 2172588) for obtaining entomopathogenic drugs, with high productivity in deep culture. The strain is active against locust larvae and egg-pods.

There is known strain Beauveria bassiana IC 1530-25-1 (patent of the Russian Federation 2704859) for the production of a biological product against the Colorado potato beetle, fungal pathogens and stimulation of potato growth during the period of vegetation.

There is known strain Beauveria bassiana SF 127-13 (application for patent of the Russian Federation 2015109186), which is the active principle of the entomopathogenic drug.

There is known a composition for combating larvae of click beetles (patent of the Russian Federation 2311778), containing a solid carrier and a microbiological source of lysine, as well as a mother culture of the entomopathogenic fungus Beauveria bassiana F-65 and succinic acid. Known microbial composition for combating the larvae of click beetles (patent of the Russian Federation 2311768), containing a solid carrier and a microbiological source of lysine, as well as an equal volume mixture of mother cultures of entomopathogenic fungi Beauveria bassiana F-65 and Metarrhizium anisopliae var. anisopliae F-596 and succinic acid.

A common disadvantage of the above mentioned inventions is the limited or indeterminate spectrum of insect pests against which the use of foregoing strains of Beauveria bassiana is effective. At the same time, it is known that the breadth of the spectrum of affected insects refers to strain-specific properties.

### OBJECTIVE AND SUMMARY OF THE INVENTION

The objective of the claimed invention is to isolate a new natural entomopathogenic fungus strain that infects a wide range of insect pests, including wheat thrips, whiteflies, cotton bollworms, green leafhoppers, wheat aphid, pea aphids, melon aphids, potato aphids, cabbage moths, tortricid onion moth, potato moth, corn borer, sunn pest, codling moth, grape fruit moth, Colorado beetle larvae, click beetle, corn sawfly and other insect pests, as well as spider mites, and compatible with at least some chemical pesticides, which would allow for joint processing without loss of viability of the biological agent.

This objective is achieved thanks to the isolation and use of the Beauveria bassiana 17HA strain deposited in the National Bioresource Center All-Russian Collection of Industrial Microorganisms NRC “Kurchatov Institute”—GosNIIGenetika (address: 1st Dorozhny proezd, 1, 117545 Moscow, Russia) under registration number VKPM F-1526, deposited date 19 Aug. 2020. The strain was isolated from the larvae of the Colorado potato beetle of third instar. The species affiliation of the culture was determined based on the results of sequencing of the nucleotide sequence of ITS-D1/D2 rDNA, at the Federal State Budgetary Institution “Go sNIIGenetika”.

### PREFERRED EMBODIMENTS OF THE INVENTION

The claimed strain has the following characteristics.

Cultural-morphological and physiological-biochemical signs. At a temperature of 25° C. on potato-dextrose agar and Sabouraud's medium, when sieving the suspension of conidia, colonies visible to the naked eye appear on the third day of growth and have a diameter of no more than three mm Colonies grow quite slowly, reaching a diameter of 38-42 mm by 14 days of growth. The colonies are white, velvety, rather dense, adjoining the surface of the medium, the mycelium grows into the agar to a shallow depth; colonies become mealy with age. The colonies are white, regardless of the age of the culture, the reverse side is yellowish. The strain does not produce soluble pigments; the release of exudate is also not typical.

The Beauveria bassiana 17HA strain has the following micromorphological signs. Vegetative hyphae are septate, branched, colorless, with smooth walls, 1-2 μm wide. Conidiophores are solitary, but collected in dense whorls of 4-5 or more pieces, with an expanded base and a thin elongated top with a zigzag indeterminate rachis, formed laterally on aerial hyphae. Conidia are colorless, non-septate, with thin smooth walls, have a size of 2-3 μm, a shape close to spherical, sometimes with an inconspicuous notch at the base. During surface growth, aerial conidia are aggregated into spherical clusters among aerial mycelium and have a highly hydrophobic shell. Under deep cultivation of the strain, the formation of blastospores occurs, the shape of which depends on the composition of the nutrient medium and the age of the culture and can be elongated (almost rod-shaped), oval, lemon-shaped, round, similar to coffee beans.

Physiological-biochemical signs.

The strain is an aerobe, a saprotroph, and upon contact with insects, it is an active entomopathogen. The optimum temperature for growth is 28° C., while the strain can grow on nutrient media in the temperature range from +12 to +35° C. Optimum pH values are in the range of 6.7-7.5, growth is observed at pH values from 3.0 to 9.0. It utilizes glucose, fructose, sucrose, maltose, glycogen, starch, glycerin, polysorbates 20-80, natural fats of vegetable and animal origin as a source of carbon and energy.

Beauveria bassiana 17HA can use ammonium salts, nitrates, urea, organic nitrogen in the form of amino acids and peptides as a source of nitrogen.

Reproduction of a microorganism can be carried out in several ways. In the surface method, conidia and mycelium of the strain Beauveria bassiana 17HA are applied to the surface of the nutrient medium in Petri dishes, microbiological mattresses or in any microbiological dish with a large working surface area, then the biomass is distributed over the surface of the medium with a Drygalsky spatula and the vessels are incubated in a thermostat at 27° C.

In the submerged culture method, the conidia of the strain germinate and form mycelium in a liquid nutrient medium in conical Erlenmeyer flasks, either in shake flasks or in a fermenter. When cultivating conidia and mycelium of the strain in flasks, shakers and incubators are used. The flasks filled with nutrient medium by ¼-⅓ of their volume are inoculated with aliquots of strain biomass from a solid nutrient medium, placed on a shaker-incubator and shaken at 28° C. for 3-5 days. When cultivating in a fermenter, the nutrient medium in the culture vessel is inoculated with daily mycelium of the strain obtained in flasks in way as described above.

As a nutrient medium for deep cultivation, Czapek-Dox medium with yeast extract is suitable (composition, g/l: sucrose—30.0; sodium nitrate—3.0, magnesium sulfate—0.5, potassium chloride—0.5, sulfate iron (II)—0.01, potassium hydrophosphate—1.0, yeast extract (DE)—0.5; distilled water to 1 l; pH 7.0) or Sabouraud's medium (composition, g/l: glucose—40 g/l, peptone—10 g/l, tap water up to 1 l; pH 7.0).

### INDUSTRIAL APPLICABILITY

Strain Beauveria bassiana 17HA is a pathogen for wheat thrips, whitefly, cotton bollworm, green leafhopper, cereal aphid, pea aphid, melon aphid, potato aphid, cabbage moth, onion moth, potato moth, corn moth, stem moth, sunn pest, apple codling moth, grape fruit moth, Colorado potato beetle larvae, click beetle, grain sawflies and other insect pests, as well as spider mites. At the same time, the bees do not show sensitivity to this strain.

Blastospores of the Beauveria bassiana 17HA strain remain viable in tank mixtures with such fungicides as Ferazim, KS (active ingredient—carbendazim) and Scalpel, KS (active ingredient—flutriafol), as well as herbicides based on chlorsulfuron.

The invention is illustrated by the following examples:

### EXAMPLE 1

A large-scale field experiment to identify the effectiveness of the claimed strain of Beauveria bassiana 17HA was carried out in 2017 in Uzbekistan in the Urtachirchik district of the Tashkent region on the “QURBONALI AGRO” farm on cotton crops in the fight against cotton bollworm, aphids and spider mites. For processing was used a deep culture of the fungus, obtained on Czapek-Dox medium with yeast extract, with a titer of 1×10⁸ blastospores/ml and a consumption rate of 3 l/ha. The introduction of the biological agent was carried out by spraying (the flow rate of the working fluid was 300 l/ha). The area of experimental plots was 1 hectare for each variant of the experiment in triplicate. Accounting for the number of pests was carried out before the treatment of plants (preliminary accounting), then on days 3, 7 and 14 after spraying. The results are presented in Table 1. The biological effectiveness of Beauveria bassiana 17HA in the fight against cotton bollworm, aphids and spider mites on cotton was 80.4%, 84.9% and 91.6%, respectively, which indicates a high degree of pathogenicity of the strain Beauveria bassiana 17HA in relation to various insects and mites—pests of plants and the absence of a pronounced specialization.

### EXAMPLE 2

Field tests of the effectiveness of Beauveria bassiana 17HA were carried out in 2017 in the Rostov region on onions against tortricid onion moth larvae and on potatoes against potato moth caterpillars. For processing was used a deep culture of the fungus, obtained on Czapek-Dox medium with yeast extract, with a titer of 1×10⁸ blastospores/ml and a consumption rate of 3 l/ha. In both cases, 2 treatments were carried out during the growing season by spraying with a working fluid consumption rate of 300 l/ha with an interval of 10 days. Accounting for the number of pests was carried out before the treatment of plants (preliminary accounting), then on days 3, 7 and 10 after spraying. The results are presented in Tables 2 and 3. In both cases, high rates of effectiveness of the use of Beauveria bassiana 17HA in controlling the number of tortricid onion moth and potato moth larvae were achieved.

TABLE 1
Biological effectiveness of Beauveria
bassiana 17HA against cotton
bollworm, aphids and spider ticks on cotton.

| | Cotton bollworm | | | | | | |
|---|---|---|---|---|---|---|---|
| | Average number of | | | | | | |
| | pests per plant, ind. | | | | Biological | | |
| | | After processing, | | | efficiency by | | |
| Experi- | Before | by days | | | days of | | |
| ment | pro- | of accounting | | | accounting, % | | |
| Variant | cessing | 3 | 7 | 14 | 3 | 7 | 14 |
| B. | 9.7 | 4.6 | 2 | 1 | 47.8 | 72.4 | 80.4 |
| bassiana | | | | | | | |
| 17HA | | | | | | | |
| Control | 9.9 | 9 | 7.4 | 5.2 | — | — | — |
| (no pro- | | | | | | | |
| cessing) | | | | | | | |

Aphids

| Average number of | | | | | | |
|---|---|---|---|---|---|---|
| pests per plant, ind. | | | | Biological efficiency | | |
| Before | After processing, by | | | by days | | |
| pro- | days of accounting | | | of accounting, % | | |
| cessing | 3 | 7 | 14 | 3 | 7 | 14 |
| 19.8 | 10.4 | 4.1 | 1.9 | 40.6 | 73.4 | 84.9 |
| 19 | 16.8 | 14.8 | 12.1 | — | — | — |

Spider tick

| Average number of | | | | | | |
|---|---|---|---|---|---|---|
| pests per 1 leaf, ind. | | | | Biological efficiency | | |
| Before | After processing, by | | | by days of | | |
| pro- | days of accounting | | | accounting, % | | |
| cessing | 3 | 7 | 14 | 3 | 7 | 14 |
| 19.4 | 11.5 | 4.7 | 1.3 | 40.1 | 72.5 | 91.6 |
| 20 | 19.8 | 17.6 | 16 | — | — | — |

TABLE 2
Biological efficacy of Beauveria bassiana 17HA
against onion moth on onions.

| | First processing | | | | | | |
|---|---|---|---|---|---|---|---|
| | Average number of | | | | | | |
| | caterpillars per plant | | | | | | |
| | | After | | | Decrease in the number | | |
| | | processing, | | | relative to the original, | | |
| | | by days of | | | adjusted for control, by | | |
| Experiment | Before | accounting | | | days of registration, % | | |
| Variant | processing | 3 | 7 | 10 | 3 | 7 | 10 |
| B. bassiana | 3.2 | 2.7 | 2.2 | 1.4 | 29.0 | 48.9 | 72.0 |
| 17HA | | | | | | | |
| Control | 3.2 | 3.8 | 4.3 | 5.0 | — | — | — |
| (no pro- | | | | | | | |
| cessing) | | | | | | | |

Second processing

| Average number of | | | | | | |
|---|---|---|---|---|---|---|
| caterpillars per plant | | | | Decrease in the | | |
| | After processing, | | | number relative to the | | |
| Before | by days of | | | original, adjusted for control, | | |
| second | accounting | | | by days of registration, % | | |
| processing | 3 | 7 | 10 | 3 | 7 | 10 |
| 1.4 | 1.1 | 1.0 | 0.9 | 79.3 | 83.4 | 85.8 |
| 5.0 | 5.3 | 6.0 | 6.3 | — | — | — |

TABLE 3
Biological efficacy of Beauveria bassiana 17HA
against potato moth on potatoes.

| | First processing | | | | | | |
|---|---|---|---|---|---|---|---|
| | Average number of | | | | | | |
| | caterpillars per plant | | | | Decrease in the | | |
| | | After | | | number relative | | |
| | | processing, | | | to the original, | | |
| | | by days of | | | adjusted for control, by | | |
| Experiment | Before | accounting | | | days of registration, % | | |
| Variant | processing | 3 | 7 | 10 | 3 | 7 | 10 |
| B. bassiana | 3.4 | 2.7 | 2.2 | 1.4 | 32.5 | 51.1 | 73.1 |
| 17HA | | | | | | | |
| Control | 3.4 | 4.0 | 4.5 | 5.2 | — | — | — |
| (no pro- | | | | | | | |
| cessing) | | | | | | | |

Second processing

| Average number of | | | | | | |
|---|---|---|---|---|---|---|
| caterpillars per plant | | | | Decrease in the number | | |
| | After processing, | | | relative to the original, | | |
| Before | by days of | | | adjusted for control, by | | |
| second | accounting | | | days of registration, % | | |
| processing | 3 | 7 | 10 | 3 | 7 | 10 |
| 1.4 | 1.1 | 1.0 | 0.8 | 80.4 | 83.9 | 87.5 |
| 5.2 | 5.6 | 6.2 | 6.4 | — | — | — |

1. A strain of the entomopathogenic fungus Beauveria bassiana deposited in the National Bioresource Center All-Russian Collection of Industrial Microorganisms NRC “Kurchatov Institute”—GosNIIGenetika under the registration number VKPM F-1526 for the protection of agricultural plants against insects and ticks—plant pests.
